(19) United States
(12) Patent Application Publication
(10) Pub. No.: US 2017/0091686 A1
(43) Pub. Date: Mar. 30, 2017

(54) SYSTEMS AND METHODS TO IMPROVE THE EFFICIENCY OF COLLABORATION FOR RESEARCH PROJECTS

(71) Applicant: Mayank GOYAL, Calgary (CA)

(72) Inventor: Mayank GOYAL, Calgary (CA)

(21) Appl. No.: 15/126,719

(22) PCT Filed: Feb. 11, 2016
(86) PCT No.: PCT/CA2016/050127

§ 371 Date: Sep. 16, 2016

Related U.S. Application Data
(60) Provisional application No. 62/114,809, filed on Feb. 11, 2015.

(51) Int. Cl. G06Q 10/06; G06Q 20/10

(52) U.S. Cl. CPC G06Q 10/06313; G06Q 20/10; H04L 67/306

(57) ABSTRACT

The invention relates generally to increasing the efficiency of knowledge sharing promoting research and innovation, and more specifically to increasing the efficiency of knowledge sharing and collaboration between experts in particular fields. More specifically, an internet-based website is described wherein users can collaborate on proposed research projects or areas of study. Users register to become members on the website, and when they register they input information which may include their areas of interest, areas of expertise, educational background, current employment, past employment, etc. such that expertise in an area is matched to a research project.

### FIELD OF THE INVENTION

The invention relates generally to increasing the efficiency of knowledge sharing promoting research and innovation, and more specifically to increasing the efficiency of knowledge sharing and collaboration between experts in particular fields.

### BACKGROUND OF THE INVENTION

It is well known that medical research undertaken to evaluate new treatments, drugs and/or medical devices, moves forward through a combination of basic research and medical trials. Typically, following the basic research, a researcher or team of researchers will undertake additional research in order to evaluate the results of the basic research and where it is an objective of the research otherwise prove the safety of the treatment, drug and/or medical device. The same general principles apply to other areas of research including engineering and science.

As is known and generally speaking, the stages of medical research include a) observation of a particular pattern from a dataset that may have been collected and maintained over a period of time, b) establishing a hypothesis, c) designing a study, typically either a retrospective (eg. a case series study) or a prospective (eg. a cohort study) study and d) the use of data that is obtained to make further observations. In the case of a new treatment, once there is accumulation of sufficient data and relative maturity of technology and/or medications, trials are undertaken.

In the case of medical research that is seeking to prove the safety of a treatment, drug and/or medical device, such research will require that the research is completed in stages, typically classified into phases (eg. FDA trial phases I, II and III) depending on whether the required work is early in the research or relatively late in the research. Typically, Phase I trials are done to demonstrate safety, Phase II trials are done to demonstrate feasibility, effect size and finally Phase III studies are done that are usually Randomized Controlled Trials where the new treatment is tested head to head against the old approach to demonstrate benefit to patients.

Most medical research studies are multi-centric meaning that more than one medical facility is involved and that can involve the participations of multiple teams, researchers, physicians and patients. Such studies have multiple advantages including faster accumulation of information especially for rare conditions, a greater degree of generalizability and overall greater credibility of the research. It is also well known that undertaking and completing such trials requires significant time and money.

In most cases, the process of receiving the money required to undertake both basic medical research and medical trials involves one or more researchers to write various grant applications to funding agencies. Many of these funding agencies are government agencies that utilize tax dollars to fund the research. Alternatively, or in combination, private companies such as the company that manufactures a drug or device sponsors the medical research and/or medical trial. Still further, philanthropic agencies, such as the Bill and Melinda Gates foundation, may also fund research that may be of particular interest to that foundation.

The process of initiating medical research (both basic and clinical) is slow for multiple reasons. Some of these are outlined below.

Generally, the demand for funding by researchers far outstrips the available supply of public and private funds for the research. Moreover, the general trend of the competitiveness for funding continues to increase.

In addition, most funding agencies have a complex process of application, review and funding. These will often include fixed dates for submission, complex submission formats and a slow evaluation process. With most agencies, once grant applications have been submitted, they are typically vetted by so called experts via a condensed and intensive process where the experts travel to a central location where they may spend several days discussing and approving or denying grants. Importantly, while the grant reviewers are typically experts within their specific field, given the complexity of the various fields of research including medicine in general, they very often don't precisely have the expertise in the topic of a particular grant.

Moreover, as noted above, the complete writing, submission and approval process is very slow. Under typical circumstances, the time from the submission of the first grant application to the grant being accepted is very often more than 2 years. Moreover, subsequent to a grant being approved, there are often various complexities related to budgets, and release of monies that can delay and/or affect the start of a research project.

In addition, and following grant approval, a further complicating step of medical research is getting the various participating centers formally engaged to participate in the research. This has two critical steps including a) have one or more legal contracts related to the obligations of the principal investigators running the research/trials and the center wanting to participate in the trial and b) establishing and involving Institutional Review Boards (IRBs) that every major center is expected to have to provide guidance and permissions related to the ethical aspects of the medical trials.

As is known, as the above steps involves lawyers on both sides as well as boards that operate within fixed timeframes, these steps can also be time consuming

Still further, for industry funded trials there are a few additional considerations. For example, a company will generally only fund only research that if successful would increase the sales of the particular drug/device in question. If a business case cannot be made it is quite difficult to obtain industry funding. In addition, company/industry sponsored trials are designed and conducted in such a way that if successful, the research results in Food and Drug Administration (FDA) approval. As such, any involvement of the FDA adds further complexity to the process.

In addition, very often from a patient point of view and/or from a medical research point of view, the more important question is whether a particular procedure (as opposed to a particular device) is beneficial. In many cases, for the same procedure, more than one device may be available that is manufactured by industry competitors. In these cases, industry finds it very difficult in a short period of time to find a way to work together to collectively move the science and the field forward such that if the trial is funded by one company, they often impose restrictions to the use of only their product in the trial.

FDA rulings also add complexity. Generally, the FDA wants ‘pure’ data related to a particular device before granting approval. While this is desirable in some respects to the extent that one does not want to mix the results of two disparate drugs and devices and grant approval to both. However, on the other hand, if the results of the two are quite similar, one does not want to delay the whole approval process just because the individual companies lack sufficient commitment and/or resources to do only one drug/device trial. This is ultimately important to the extent that the patient may not necessarily care.

Other implications of a slow and/or inefficient research process relates to the effect on the business case for the research. Generally, innovation is guided by the ‘business case’ and the overall costs of taking an idea to approval, sale and revenue generation. The longer and more time consuming this process is, the lower is the motivation for innovators, venture capital etc.

Rising costs are also a significant factor in affecting business decisions around undertaking research. Ultimately, the company manufacturing a drug/device has to make profit for the whole industry model to be sustainable. The longer the whole process takes, the higher will be the cost after it is approved.

Patient harm or lack of patient benefit is also a factor. Certain harmful procedures, drugs may continue in the absence of well conducted research. For example, giving high dose steroids for spinal cord injury is known to have a negative affect notwithstanding earlier research suggesting its benefit. Also, certain drugs/drug treatments that could have benefit may not be used for years for the want of adequate trial data.

Generally speaking, it is known that the involvement of a larger pool of opinions and views in considering a problem is more likely to reach a “best solution” as compared to the same problem being considered by a smaller group that may spend more time considering the same problem. That is, in a similar way that research results from a larger data set from multiple centers provides greater credibility and generalizability to the results, the opinions and views of a larger pool is more likely to reach the “best solution”. In addition, having access to a wider pool of opinions can result in greater efficiency in obtaining a best solution. That is, it is known that if an individual has a particular question or issue that they do not have an answer to, posing that question on an internet forum can quickly and efficiently provide an answer to that question as the question may be considered by individuals who may be experts in the field of the question and/or who may provide validation to the answer provided by others thus promoting the legitimacy of an answer.

In view of the foregoing, there has been a need for systems and processes that generally enable greater efficiency in the overall process of initiating and reviewing the merits of various research projects. In particular, there has been a need for the ability for a wider pool of experts to consider and evaluate research projects within a forum that enables critical evaluation and involvement of those experts in the review and validation of those projects.

Furthermore, there has been a need for an efficient and iterative process that allows a pool of experts to assist in contributing to the design and improvement of a particular research project. That is, in most cases, the grant application process is one-way in that the agency either says yes or no with regards to approval. As such, there has been a need for systems and methods that allow for two-way interaction between a principal investigator, experts and funding groups that can more efficiently enable iterative feedback and discussions to occur that have the objective of improving the design of the study.

Further still, there has been a need for systems and methods that can more efficiently bring a more divergent or broader scope of expertise and opinions together. That is, with some studies there may be various issues related to systems of care, geography, ethnic variability of disease etc. that could benefit from a broader perspective when considering and designing a study.

In another aspect, and in the same context, there has been need for systems and processes that enable greater efficiency in the collaboration of experts to consider, discuss and propose solutions to various problems in other areas of study. For example, collaboration in essentially all areas of technical or scientific study, artistic study, political study etc. would benefit from systems that likewise improve the efficiency of collaboration when seeking solutions to various problems. Such systems may also be used for the benefit of teaching and involving students in the process of collaboration and iterative learning.

### SUMMARY OF THE INVENTION

Briefly, the invention is directed to a system and method for sharing knowledge amongst qualified users. While the following description is generally written in the context of medical research, it is understood that the concepts and functions described herein can be applied to other areas of study.

More specifically, the invention comprises an internet-based website wherein users can collaborate on proposed research projects or areas of study. Users register to become members on the website, and when they register they input information which may include their areas of interest, areas of expertise, educational background, current employment, past employment, etc. For example, a physician may register and indicate their expertise to be neurology, and sub-expertise to be strokes.

A first user (eg. a principal investigator or “PI”) can post a proposed research project in a given format, which may be limited to a certain number of words (for example 1000 words), and/or may require specific sections to be filled out. For example, a background, a 1 sentence summary, the motivation, the proposed duration of research, the funding requirements, equipment and facility requirements, questions, areas where specific input is sought, etc.

After posting, other registered users can log in and view the proposed research project. The project may be open such that any registered user may view the project, or it may be closed and limited to users having certain qualifications, or upon invite only.

After a project has been posted, the PI may solicit input from specific users or groups of users, or general users. Upon receipt of input from various users, the PI can amend the proposal.

In addition, the project can be established to enable collaboration where users can provide comments and/or post questions about the research project. The comments and questions may be posted in a forum available to all users, or can be sent privately to the PI. The PI and other users may provide comments and/or answers back, or pose further questions. During collaboration, the proposal can be amended.

In addition, each proposal may be scored by users where each user can provide a score or rating on the merits of the project through simple voting and/or through a questionnaire.

In one aspect, the invention provides a system for enhancing collaboration between users of an internet enabled website allowing multiple users to interact with the website, the system including:a computer system including at least one input system and at least one graphical output system, the computer operatively connected to the internet and including non-transitory computer readable medium encoded with instructions supporting:a project registration module enabling a principal investigator (PI) to describe a project including at least question to be answered;a collaboration authorization module enabling the PI to establish collaboration criteria including at least one area of expertise and/or area of interest desired by the PI to potentially answer the at least one question and to enable authorized users to gain access to the project description when authorized by the PI; and a project collaboration module enabling authorized users to review the project and provide feedback to the PI with regards to the at least one question to be answered.

In one embodiment, the system includes an authorized user registration module enabling an authorized user to register with the system and enabling an authorized user to define one or more areas of expertise and/or areas of interest.

In another embodiment, the collaboration authorization module matches an area of expertise or area of interest of an authorized user with an area of expertise or area of interest required by the PI and when a match is obtained, the collaboration authorization module grants the authorized user access to the project description.

In one embodiment, the collaboration criteria of an authorized user includes any one of or a combination of:technical expertise defined by one or more technical keywords in relation to a technical discipline;financial expertise defined by one or more financial keywords in relation to a financial discipline;commercial expertise defined by one or more commercial keywords in relation to a products or services discipline.

In one embodiment, an authorized user is a funding group including any one of or a combination of a government funding agency, a private company, a philanthropic organization, an investor, an investor group and one or more individuals.

In another embodiment, the project collaboration module supports at least one phase of collaboration between the PI and at least one authorized user where the at least one phase of collaboration includes any one of or a combination of:a project design phase enabling iterative collaboration between the PI and at least one authorized user for the purpose of modifying the description of the project and/or the one or more questions to be answered;a project funding phase enabling iterative collaboration between the PI and at least one authorized user for the purpose of pledging funds to complete the project and;a project execution phase enabling iterative collaboration between the PI and at least one authorized user for the purpose of executing a number of steps to complete the project.

In another embodiment, during the project design phase and/or during the project funding phase, at least two authorized users as authorized by the PI are enabled to provide feedback on pre-defined merits of the project selected from any one of or a combination of a) a yes or no vote on the merits of the project and b) a questionnaire related to aspects of the project to create a ranked list of projects.

In yet another embodiment, the website is enabled to list a plurality of individual projects and the project collaboration module includes a ranking module enabling authorized users to vote up and/or vote down individual projects as listed in order to create a ranked list of projects.

In a further embodiment, the project registration module includes a template for defining the project including a completable section for defining access parameters to authorized users as determined by the PI.

In one embodiment, the project registration module includes a video description module.

In another aspect, a system enabling targeted financial contributions by a donor to an organization supporting multiple projects is provided, the system including an internet enabled website allowing multiple users to interact with the website, the system comprising:a computer system including at least one input system and at least one graphical output system, the computer operatively connected to the internet and including non-transitory computer readable medium encoded with instructions supporting:a project description module supporting the description of a plurality of projects supported by the organization, the project description module accessible by users accessing the system website and allowing users to review the content of individual projects within the project description module; and a financial contribution module operatively linked to the project description module enabling a user to make a financial contribution to an individual project within the project description module.

In one embodiment, the website is enabled to list a plurality of individual projects and the project description module includes a ranking display of individual projects where the ranking display displays the input of authorized users who have provided input on the merits of individual projects.

In another embodiment, authorized users are authorized to provide feedback on pre-defined merits on a list of projects to create a ranked list of projects, where feedback is provided by any one of or a combination of a) a yes or no vote on the merits of the project and b) a questionnaire related to aspects of the project.

### BRIEF DESCRIPTION OF THE DRAWINGS

Various objects, features and advantages of the invention will be apparent from the following description of particular embodiments of the invention, as illustrated in the accompanying drawings. The drawings are not necessarily to scale, emphasis instead being placed upon illustrating the principles of various embodiments of the invention. Similar reference numerals indicate similar components.

FIG. 1 is an overview of a project collaboration system in accordance with one embodiment of the invention showing the general relationship between parties.

FIG. 2 is an overview of a project registration module of a project collaboration system in accordance with one embodiment of the invention.

FIG. 3 is an overview of a project creation module of a project collaboration system in accordance with one embodiment of the invention.

FIG. 4 is an overview of a user registration module of a project collaboration system in accordance with one embodiment of the invention.

FIG. 5 is an overview of a project progress module of a project collaboration system in accordance with one embodiment of the invention.

FIG. 6 is an overview of a project communication module of a project collaboration system in accordance with one embodiment of the invention.

FIG. 7 is an overview of a project revenue module of a project collaboration system in accordance with one embodiment of the invention.

FIG. 8 is a representative example of the high level functionality of the project collaboration system in accordance with one embodiment of the invention.

### DETAILED DESCRIPTION OF THE INVENTION

### Scope of Language

All terms have definitions that are reasonably inferable from the drawings and description. Language used herein is to be interpreted to give as broad a meaning as is reasonable within the inventive concepts described herein.

It is to be understood that prior art cited during prosecution of the application may not have been identified as of the filing date and that various amendments may be introduced during prosecution that require amendment of terms to provide clarity to the distinctions between the subject invention and that prior art including defining specific relationships between elements that require additional temporal or spatial explanation to provide these distinctions.

In cases where prior art may be more complex than the subject invention, reciting more elements than the subject invention, the absence of those features may be relied upon as inventive and while the Applicant may recite fewer elements as an inventive combination, it is understood that additions and modifications to that combination of elements may still be made.

### Introduction

Various aspects of the invention will now be described with reference to the figures. For the purposes of illustration, components depicted in the figures are not necessarily drawn to scale. Instead, emphasis is placed on highlighting the various contributions of the components to the functionality of various aspects of the invention. A number of possible alternative features are introduced during the course of this description. It is to be understood that,according to the knowledge and judgment of persons skilled in the art, such alternative features may be substituted in various combinations to arrive at different embodiments of the present invention.

### Overview

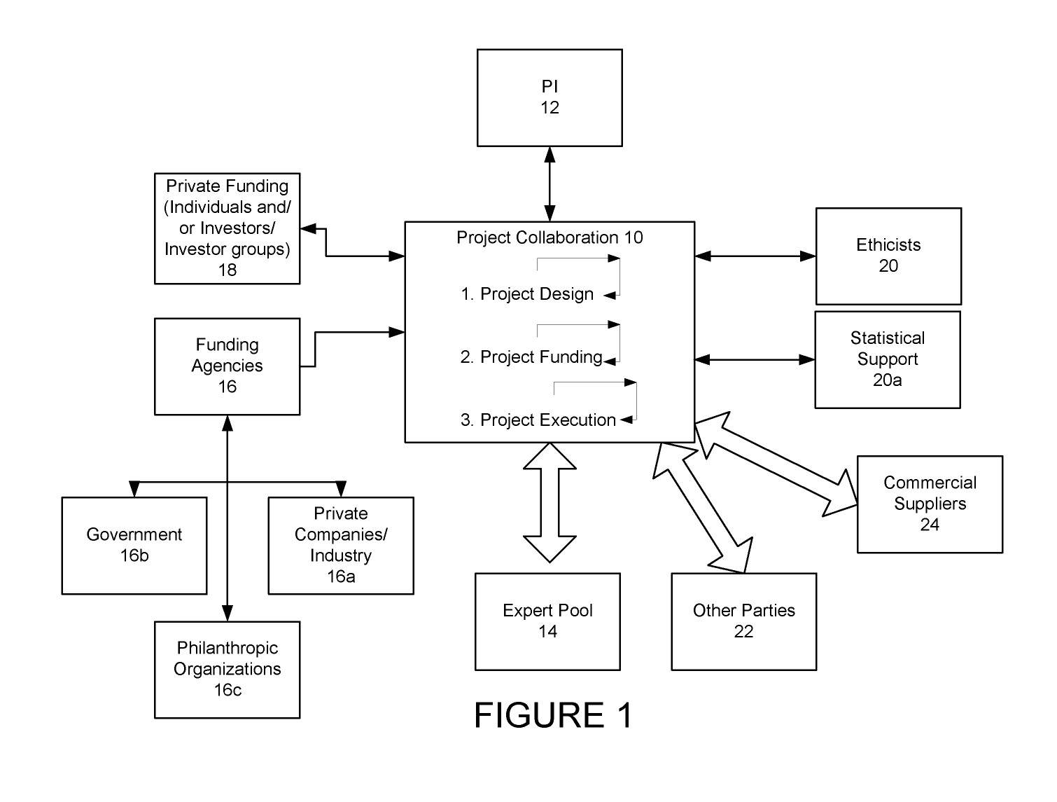

In accordance with the invention, systems and methods for enabling collaboration between parties is provided. As shown in FIG. 1, various parties can interact with a project collaboration system 10 to provide collaborative input to a defined project. Generally, a project sponsor or principal investigator (PI) 12 will upload a project to the project collaboration system and enable input from other parties to review and provide their input to the project. The other parties generally include experts 14, funding agencies 16 including private funding groups 16a, government funding groups 16b and philanthropic funding groups 16c, ethicists 18 (if required), statistical support groups 18a (if required) other private funding groups (including individuals and/or investors/investor groups) and other parties 22 (eg. individuals) who may have an interest in participation with the project. In addition, the project collaboration module may be made available to commercial suppliers 24 of products and services who may have a commercial interest in participating with the project. Still further, other potential collaborators could include cultural experts, social scientists, computer programmers and information technology experts as well as many other different persons or groups having a particular expertise.

The overall objectives and operation of the system and the definition of the parties and their involvement with the project collaboration system is described by way of the following general example. Additional objectives and functionality of the system are further described in subsequent sections of this description. It is understood that the example described is not intended to be limiting in terms of the principles discussed therein and that while the example is written in the context of a medical project, the principles can be applied to any nature of collaborative project in the arts and sciences.

Initially, a project sponsor (generally referred to herein as a principal investigator PI12) creates a research project for which they wish to have reviewed for the primary objectives of obtaining private and/or government funding to complete. The research project may have a fundamental commercial objective or may have a basic non-commercial objective. For example, in the case of a project having a commercial objective, the researcher may be an orthopedic surgeon who wishes to evaluate and potentially develop a new hip replacement implant. The scope of the project requires both basic research in the design and engineering of the proposed implant, basic laboratory and non-clinical research to evaluate the engineered design and non-clinical questions of the implant, and thereafter, if the basic research is successful, a full clinical evaluation of the implant across multiple centers for the ultimate objective of full commercialization of the proposed device.

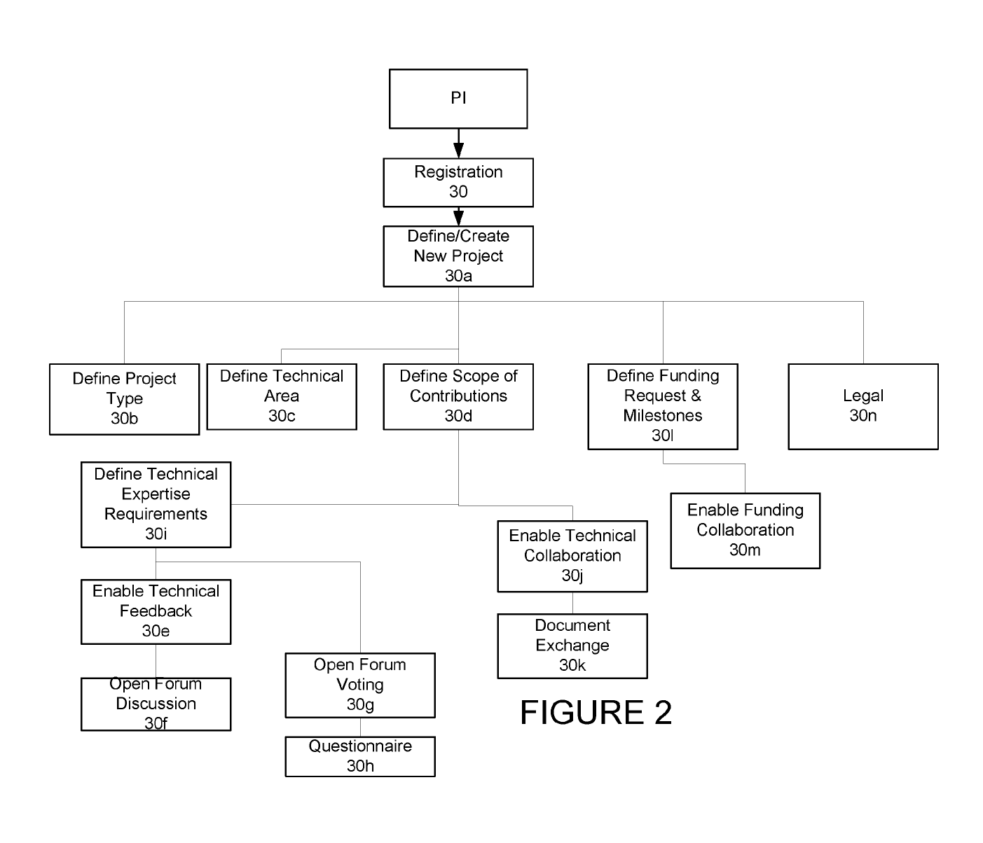

As shown in FIG. 2, the PI accesses the project collaboration system through a registration process and initially defines the type of new project being created 30a. Generally, the type of project 30b will be defined as any one of or a combination of a) a basic research, b) a clinical research, c) a technical collaboration and d) a commercial project. In addition, the technical area 30c will be defined. As appropriate for the type of project that has been defined, the sponsor will enable and/or activate different functions for the various parties to participate with the project as well as the type of participation depending on the interests and/or expertise of the other parties.

For example, as shown in FIG. 2, the sponsor may enable technical feedback 30e that may involve any one of or a combination of an open forum discussion 30f, open forum voting (up or down voting arrows) and a questionnaire (simple or complex). Generally, technical feedback will be limited to those registrants (explained below) who have established themselves as having a particular level of expertise 30i that may be established by the P1.

In addition, a PI may wish to enable technical collaboration in which case, the system would be activated to enable technical collaboration such as creating a project workspace where documents could be up and downloaded and otherwise exchanged 30k. Technical collaboration will generally be an iterative process where a meaningful exchange of information is enabled for the purposes of improving the project.

Further, in the case of a commercial project, the PI may define funding requests and milestones 30l and otherwise enable funding collaboration 30m.

Access to a legal module 30n that may provide the legal implications and rules of using the site may be required depending on the type of project that is defined.

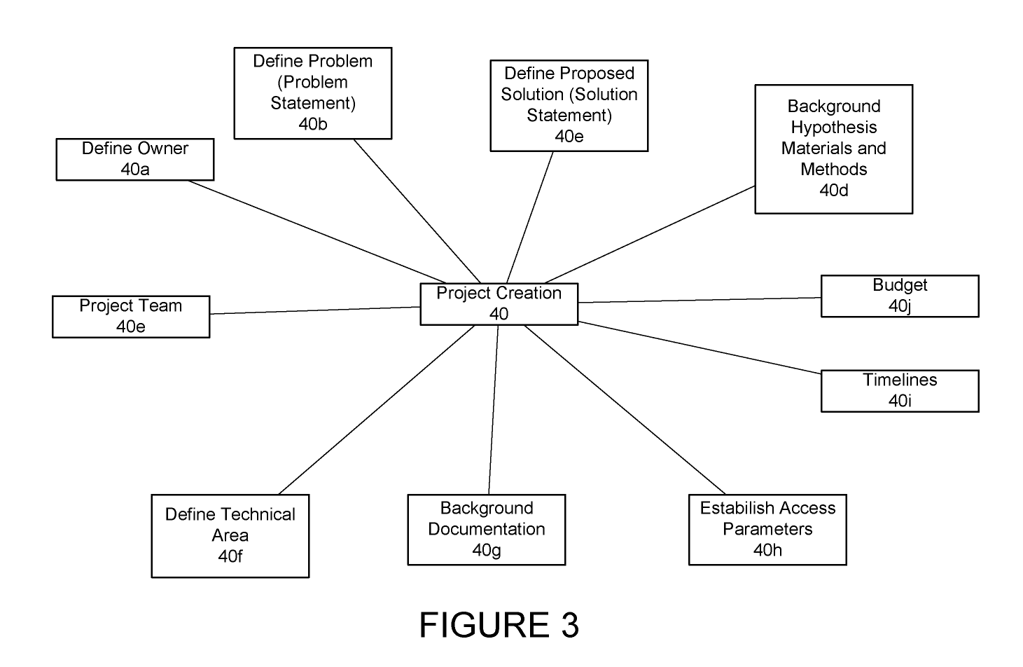

After a project has been defined, details of the project are input by the PI through a project creation module 40 as shown in FIG. 3. Generally, the project creation module enables the PI to provide the necessary details of the project in a succinct format as defined by various templates. These will typically include a definition of the project owner 40a, a problem statement 40b, a solution statement 40c, the background, hypothesis and materials and methods required to seek the solution 40d, the project team 40e, and the technical area 40f. Project timelines 40i and project budget 40j may also be input. In addition, the project creation module may enable appropriate background documentation 40g to be uploaded. Access to each of the above information may only be granted to specific users as may be determined by the sponsor through establishing access parameters 40h.

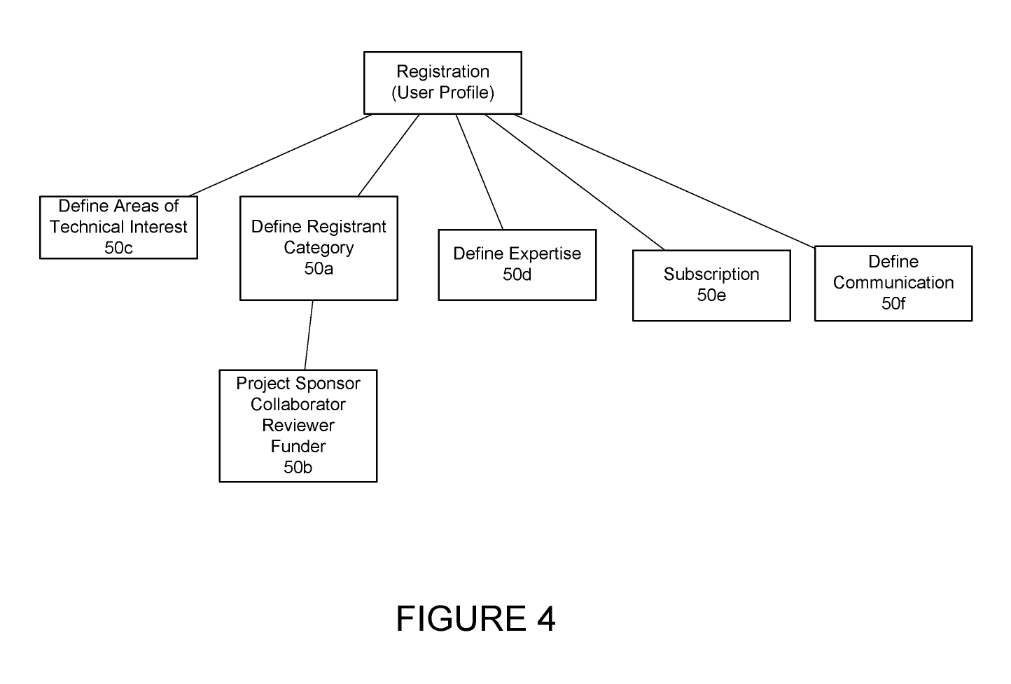

After a sponsor has defined a project, other parties (i.e. users) including funding agencies, experts, collaborators and/or investors may gain access to a project by registering with the system. As shown in FIG. 4, different parties may register with the system through a user registration module 50. Generally, each registrant will define their category of interest 50a, for example, they are a project sponsor, technical collaborator, technical reviewer or have a funding interest 50b. Each user will define their areas of technical interest 50c and, as appropriate, define their expertise 50d.

Further, a user may be required to activate a subscription to enable their participation. Subscriptions 50e may be based on a number of models depending on the type of user. For example, project sponsors/PIs seeking technical collaboration or non-commercial funding may have no subscription fee whereas project sponsors/PIs seeking funding for commercial projects may be required to pay an initial and/or ongoing fee as well as a commission on funding pledged.

The desired level of communication 50f that a user wishes to receive via email and/or other communication means (including phone, fax, tweets, texts, etc.) may be established as well.

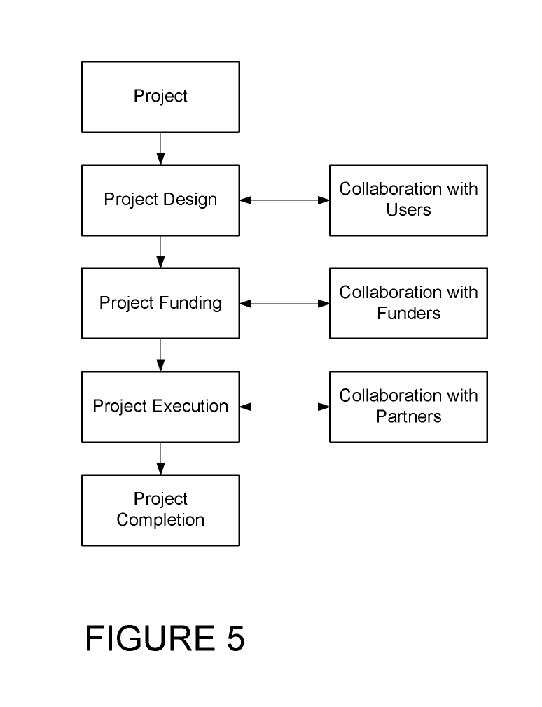

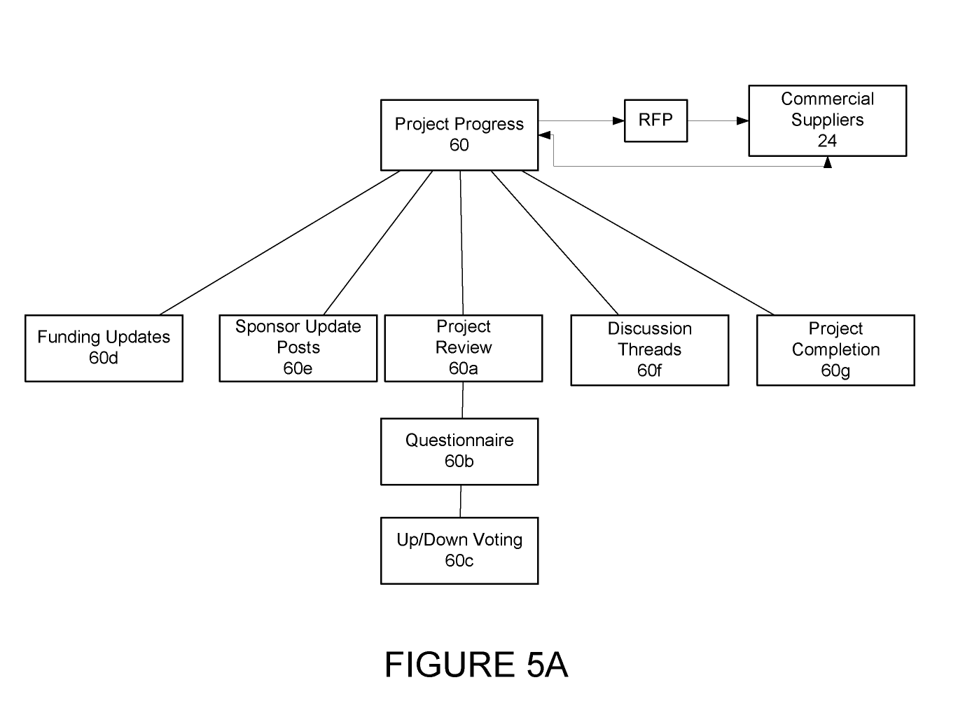

After the various users have registered with the system, a project may be activated or made available for collaboration where it will then be enabled to progress through the stages of the project as shown in FIG. 5. That is, a project will typically progress through various combinations of a) project design, b) project funding and c) project execution, where each may involve iterative collaboration with different users including technical experts, funders and/or project partners. During each stage, the system will generally enable users to provide feedback to the PI that may be used to improve the design and/or execution of the project and a forum (such as a discussion thread) to allow two-way communication between the various users. For example, a PI may communicate with any user and/or technical users may interact with one another with or without involvement of the PI.

The progress of a project may be tracked depending on the type of project that has been established as shown in FIG. 5A. As can be appreciated, the system is intended to support many projects and the different types of projects simultaneously, such that the progress of the projects will be anywhere from newly created through to fully completed projects.

The degree of visibility of a project will be dependent on the type of project. For example, technical collaboration projects may have lower visibility at the highest levels of the website, whereas meritorious and/or popular basic research, clinical and/or commercial projects that are receiving positive reviews from a technical and/or funding perspective may be more visible.

As noted above, in the case of a research project seeking funding, the system will generally operate to enable appropriate users to consider and provide feedback on the merits of a project.

In the case of the orthopedic surgeon who has defined a project as a combination of basic and clinical research as well as a commercial project, the project would generally have been defined within various technical areas including, for example, “medicine”, “orthopedics”, “hip”, “medical device”, “biomaterials”, “surgical technique”, “titanium”, etc. The surgeon may be seeking technical collaboration, for example from a biomaterials expert as well as feedback on the merits of the project from a pool of orthopedic surgeons who are hip replacement specialists.

As such, as the surgeon is creating the project, appropriate permissions may be granted to those individuals who have the expertise to potentially collaborate. In addition, permissions may be granted to those having the expertise to provide input 60a on the merits of the research and/or provide comments that may improve the design of the project.

Ultimately, and to the extent that the system, may be simultaneously hosting a number of projects relating to hip replacement implants, the system seeks to enable appropriate users to review the various hip replacement implant projects and provide both subjective and objective input on the merits of the various projects. The manner in which input can be provided can be more subjective by enabling various reviewers to simply “vote” with an up or down arrow (thumbs up, thumbs down) system 60c that will either promote or drop a project in a list of related or similar projects or may involve a more sophisticated questionnaire 60b that may provide a more objective score that may be given to a project which can move a project up or down in the ranking. Similarly, a discussion thread within the project may allow contributors to vote up or vote down posts within a discussion thread.

Ideally, however, the objective of the system is to more efficiently allow a greater number of reviewers to provide input regarding their opinion regarding the design of the project and/or the value and/or merit of a number of projects, such that funding agencies who may be contributing to the project will have a broader range and scopes of opinion regarding a number of related projects.

Importantly, the input received from a broader pool of reviewers may be highly effective in enabling more efficient decisions to be made to the extent that a smaller pool of “traditional” reviewers within a funding organization may grant significant weight to the collective opinion of a broader pool of experts. Thus, it is an objective that through the widespread implementation of the subject system, the speed and efficiency through which important funding decisions can be made can be significantly streamlined. Importantly, as can be appreciated, to the extent that large funding organizations who have significant overhead costs in running their organizations can draw on the wider and broader expertise of the internet, there is a significant potential to reduce the overhead costs associated with such organizations, such that a larger percentage of budgetary funds may be allocated to the research as opposed to the administrative costs of running the organization.

A project that has had a funding objective defined may provide a funding update 60d as a further means of providing real-world consideration as to the merits of a project. In addition, a sponsor may provide ongoing updates 60e regarding the project having regard to technical considerations, results and other project developments. Ongoing discussion threads 60f may form part of the project progress. Further, an indication of project completion (or withdrawal) 60g may also be provided.

As noted above, in various projects collaboration may also involve the input of ethicists who may be able to contribute to the improvement of the project proposal and/or to the merits of the project. Other contributors may include for example a statistical support group that may be able to contribute the design and execution phases of a project.

In one embodiment, the project collaboration module is also opened to commercial suppliers of products and services. That is, if authorized, a commercial supplier may be able to contact a PI to propose their involvement with a project for their commercial gain. In this regard, a PI may be able to access specific commercial expertise that may be beneficial in the execution of the project. Such suppliers may include for example, companies having particular products or services required to execution of the project including specific equipment or supplies. In addition, such suppliers may include service providers such as law firms who may have particular expertise required for the project such as business law and/or intellectual property expertise.

In addition, when commercial suppliers may be involved, the PI may be able to utilize the system to efficiently establish business relationships with suppliers through a request for proposal (RFP) process from suppliers. In some cases, such processes may be required by a funding agency to ensure funds are being efficiently spent.

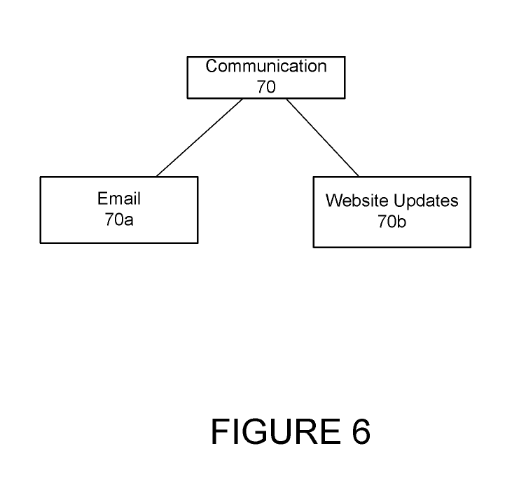

As shown in FIG. 6, the system will preferably also include a communication module 70 that allows users to control and/or manage the communications they receive from the system website. Generally, a user may select the type and frequency of emails 70a received or may simply access the website 70b for updates.

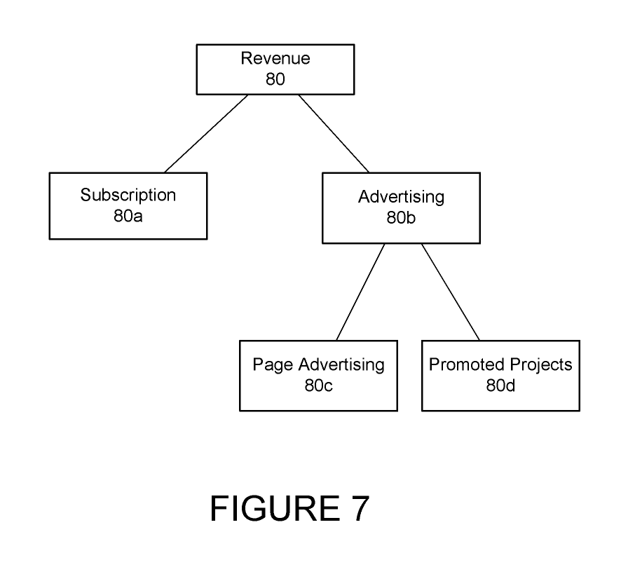

The system may be implemented in a non-commercial format or,as shown in FIG. 7, with a commercial format 80 requiring users to have subscriptions 80a to the system. In addition, the system may allow advertising 80b in various forms including traditional page advertising 80c and/or promotion of particular projects 80d within the website by payment. For example, commercial suppliers will generally be able to purchase advertising with the system and/or may be required to have a subscription to the system and a PI may be able to promote their project at different levels of the website through payments.

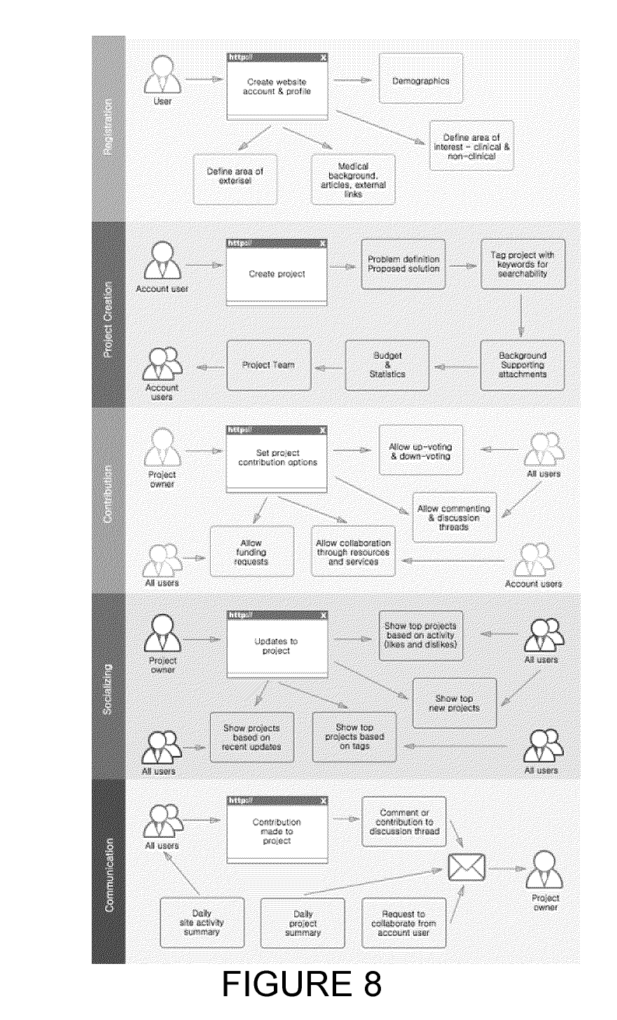

FIG. 8 is a representative example of the high level functionality of the project collaboration system in accordance with one embodiment of the invention showing the different ways in which different users may interact with the system.

Additional functionality of system is described that may be implemented as well as other benefits and advantages in improving the efficiency of collaboration and research are listed:
The system is a Web-based solution to enable a broad range of inputs from all geographical regions.
The system enables access to a wide range of participants potentially including in the case of medical research, physicians, trainees, nursing staff, healthcare administrators, representatives for ethics boards, representatives from relevant industry, representatives from funding agencies, representatives from payors.
Project submissions will preferably be limited in size utilizing standardized formats and a maximum number of words for various sections.
Project submissions may include a video submission, allowing the sponsor/PI to impart their emotion and/or passion for the project. This would enable readers to see a short video that could include aspects of the project that increase the operative understanding of the project more quickly than by written documents and drawings. Longer videos may also be part of the project as well.
Back-end administration and organization of projects may be utilized to enable a number of related projects to be considered within a particular time frame.
Back-end administration may implement particular questionnaires common to a number of related projects.
A top proposal would emerge after set periods of time. As noted, if a number of related projects have been posted at a particular time, a ranking of the proposals can be obtained by third party reviews of the merits of the respective projects by “up” or “down” voting. That is, a reviewer may review 5 projects and rank the 5 projects from 1 to 5 by either clicking on up or down arrows such that the project receiving the greatest number of up clicks is ranked first or each project is directly ranked and the highest or lowest cumulative score determines ranking. Similarly, a more detailed questionnaire may be enabled that provides a score to the projects which is used as the basis for ranking.
System has reduced likelihood of biases (that could be there from a traditional review process).
System promotes a broader field/range of collaboration.
System may enable individuals to donate monies towards a project. For example, if the budget of a trial is 1 million dollars, this could be raised by small donations from individuals and/or interested groups.
System may enable industry at any time to come in and show their financial support.
Fund raising organizations and patient advocate groups could provide financial support through targeted fund raising.
Individuals or corporations of high net worth could give targeted donations.
Conduct of trials and accountability of funds can be assisted through the system through the use of system auditors.
Other parties, including universities, may be involved to assist in funds accountability where appropriate.
Results and reporting may follow current systems.
System may enable dramatic speeding up of research review and grant process.
Involvement of a larger pool of smart people arguing and criticizing a project is more likely to yield a higher quality proposal/result.
The system may not have a fixed denominator of funds (unlike a typical funding agency).
If the community at large likes more than one proposal both or more can be funded.
A web based solution may provide a mechanism for industry partners to work with each other.
No geographical barriers (eg. a person from China can comment on a proposal from Seattle).
System could potentially free up traditional funding agencies like NIH to fund research that is more long term, felt to be very important but does not have popular support, research on rare conditions, research of massive scale (that may be beyond the capacity of the contributing individuals or industry.
Projects may be linked to a specific charitable organization. For example, one charitable organization (eg. Heart and Stroke Foundation) may list a number of projects and allow donors to select and/or evaluate the specific project they wish to support through a financial contribution. That is, when a donor wishes to make a contribution, they may be able to make a general contribution that is not targeted to any particular project or they may be prompted to review and/or identify a project that they wish to specifically support.
Projects are not limited to medical projects and could involve other projects in other scientific or artistic disciplines.
A PI may send invites to friends/colleagues with a project ID number and/or link as a means of inviting them to participate in the project.
The system may collaborate with a number of different organizations including charitable organizations, professional bodies and/or granting agencies allowing such organizations through their websites to link to one another. For example, a splash page of the system website may have logo links to different organizations and such organizations may have links back to the system website thus allowing users to review projects on the system website but also interact with an organization's website.
The system could support sponsored prizes for research. For example, an organization may promote a project and set out a challenge to the world to solve. As such, the system may support the means by which people can collaborate on a global solution and/or create sub-projects within the main project that enable collaboration.
   Collateral benefits
Problems that have no obvious champion and have no business case could also be solved this way.
A person from a sub-Saharan country could bring up a question that is unique to that part of the world. At the same time everyone from across the world is free to provide solutions and research designs.
Many types of projects can be supported and benefit from the system, including big picture societal questions such as collaboration on questions of alternative energy, population control, women's education throughout the world, political issues etc.

Although the present invention has been described and illustrated with respect to preferred embodiments and preferred uses thereof, it is not to be so limited since modifications and changes can be made therein which are within the full, intended scope of the invention as understood by those skilled in the art.

1. A system for enhancing collaboration between users of an internet enabled website allowing multiple users to interact with the website, the system comprising:
a computer system including at least one input system and at least one graphical output system, the computer system being operatively connected to the internet and the computer system including non-transitory computer readable medium encoded with instructions supporting:
a project registration module enabling a principal investigator (PI) to describe a project including at least question to be answered;
a collaboration authorization module enabling the PI to establish collaboration criteria including at least one area of expertise and/or area of interest desired by the PI to potentially answer the at least one question and to enable authorized users to gain access to a project description when authorized by the PI;
a project collaboration module enabling authorized users to review the project and provide feedback to the PI with regards to the at least one question to be answered.

2. The system as in claim 1, further comprising an authorized user registration module enabling an authorized user to register with the system and enabling an authorized user to define one or more areas of expertise and/or areas of interest.

3. The system as in claim 2, wherein the collaboration authorization module matches an area of expertise or area of interest of an authorized user with an area of expertise or area of interest required by the PI and when a match is obtained, the collaboration authorization module grants the authorized user access to the project description.

4. The system as in claim 3, wherein the collaboration criteria of an authorized user includes any one of or a combination of:
technical expertise defined by one or more technical keywords in relation to a technical discipline;
financial expertise defined by one or more financial keywords in relation to a financial discipline;
commercial expertise defined by one or more commercial keywords in relation to a products or services discipline.

5. The system as in claim 1, wherein an authorized user is a funding group including any one of or a combination of a government funding agency, a private company, a philanthropic organization, an investor, an investor group and one or more individuals.

6. The system as in claim 1, wherein the project collaboration module supports at least one phase of collaboration between the PI and at least one authorized user, wherein the at least one phase of collaboration includes any one of or a combination of:
a project design phase enabling iterative collaboration between the PI and at least one authorized user for the purpose of modifying the description of the project and/or the at least one question to be answered;
a project funding phase enabling iterative collaboration between the PI and at least one authorized user for pledging funds to complete the project and;
a project execution phase enabling iterative collaboration between the PI and at least one authorized user for executing a number of steps to complete the project.

7. The system as in claim 6, wherein during the project design phase and/or during the project funding phase, at least two authorized users as authorized by the PI are enabled to provide feedback on pre-defined merits of the project selected from any one of or a combination of a yes or no vote on the merits of the project and a questionnaire related to aspects of the project to create a ranked list of projects.

8. The system as in claim 1, wherein the website is enabled to list a plurality of individual projects and the project collaboration module includes a ranking module enabling authorized users to vote up and/or vote down individual projects as listed in order to create a ranked list of projects.

9. The system as in claim 1, wherein the project registration module includes a template for defining the project including a completable section for defining access parameters to authorized users as determined by the PI.

10. The system as in claim 1, wherein the project registration module includes a video description module.

11. A system enabling targeted financial contributions by a donor to an organization supporting multiple projects, the system including an internet enabled website allowing multiple users to interact with the website, the system comprising:
a computer system including at least one input system and at least one graphical output system, the computer system being operatively connected to the internet and the computer system including non-transitory computer readable medium encoded with instructions supporting:
a project description module supporting a description of a plurality of projects supported by the organization, the project description module being accessible by users accessing the system website and allowing users to review content of individual projects within the project description module;
a financial contribution module operatively linked to the project description module enabling a user to make a financial contribution to an individual project within the project description module.

12. The system as in claim 11, wherein the website is enabled to list a plurality of individual projects and the project description module includes a ranking display of individual projects, wherein the ranking display displays the input of authorized users who have provided input on the merits of individual projects.

13. The system as in claim 12, wherein authorized users are authorized to provide feedback on pre-defined merits on a list of projects to create a ranked list of projects, wherein feedback is provided by any one of or a combination of yes or no vote on the merits of the project and a questionnaire related to aspects of the project.
